(19) United States
(12) Patent Application Publication
(10) Pub. No.: US 2026/0068890 A1
(43) Pub. Date: Mar. 12, 2026

(54) ENDOPHYTIC MICROBIAL BIOSTIMULANTS

(71) Applicant: EVONIK OPERATIONS GMBH, Essen (DE)

(72) Inventors: Stefan GILCH, Dülmen (DE); Hans-Georg HENNEMANN, Bedburg (DE); Linda MICHEEL, Dorsten (DE); Juilia NIEWALDA, Selm (DE); Hanna-Christin LAKOWITZ, Bochum (DE); Moritz Sebastian ENGEMANN, Dortmund (DE)

(73) Assignee: EVONIK OPERATIONS GMBH, Essen (DE)

(21) Appl. No.: 19/107,840

(22) PCT Filed: Aug. 24, 2023
(86) PCT No.: PCT/EP2023/073217

§ 371 Date: Feb. 28, 2025

(30) Foreign Application Priority Data
Sep. 1, 2022 (EP) 22193372.4

(51) Int. Cl. A01N 63/20; A01N 25/10; A01P 21/00; C12N 1/20; C12R 1/01

(52) U.S. Cl. CPC A01N 63/20; A01N 25/10; A01P 21/00; C12N 1/205; C12R 2001/01

(57) ABSTRACT

The present invention relates to a composition comprising:
an isolated endophyte, wherein the endophyte is a strain of Priestia aryabhattai, and
an adjuvant
wherein the Priestia aryabhattai strain has Accession Number DSM 34352.

### FIELD OF THE INVENTION

The present invention relates to a novel isolated plant microbiome strain, in particular an endophyte, plants infected with such strains and related methods. In particular, the isolated endophyte may be used in a composition for use as a biofertilizer and/or biostimulant or for bioprotection in plants. The composition may further comprise an adjuvant.

### BACKGROUND OF THE INVENTION

In many cases, the growth and/or yield of agricultural crops is limited by the amount of nitrogen that can be used or taken up by the plant. In order to overcome this limitation, exogenous nitrogen is added to the soil before or after sowing. The nitrogen fertilizer can be of organic (e.g., urea, amino acids, manure, horn shavings) or mineral (e.g. ammonium nitrate, ammonium sulphate, potassium nitrate) origin in nature. What all these anthropogenic nitrogen fertilizers have in common is that their production, transport and/or application is energy-intensive and thus leads to a positive CO₂ balance. In particular, ammonium nitrate and urea, which are by far the most widely used nitrogen fertilizers worldwide, have a large CO₂ footprint because their common precursor ammonia (NH₃), is derived from the Haber-Bosch process. This process uses atmospheric nitrogen (N₂) and hydrogen (H₂) to synthesize ammonia (NH₃) under high temperatures and pressures. This is process thus adds to climate change and is not good for the environment.

An alternative and more sustainable method to provide plants with at least some part of the nitrogen they need is to use biofertilizers. Biofertilizers are made up of living microorganisms that are able to reduce atmospheric nitrogen and thus make it available to the plant in form of ammonium/ammonia or organically bound nitrogen. These organisms have to be applied in a metabolically active form to the seed, root or in rarer cases to the leaf.

Previous and current use of these biofertilizers apply the nitrogen-fixing microorganisms either to the surface of the seed, to the leaf or the soil. Due to this fact, the organisms are exposed to high stress from abiotic factors such as temperature, drought, pH, and wash-off from rain and high moisture. The effectiveness and efficiency of the biofertilizers is reduced. A large amount of the biofertilizers is thus needed to enable them to be effective in providing bioprotection to the plants and/or increasing yield of the crops. This increases the costs of farming.

Further, biotic factors such as high competition with the already predominant plant or soil microbiome or the presence of antibiotically active substances also reduce the survival rate of the exogenously added nitrogen-fixing microorganisms on the respective surfaces. This also reduces the efficiency of the currently available biofertilizers.

Accordingly, there is a need in the art for a means to better exploit these endophytes for use as biofertilizers and/or biostimulants to improve sustainable agriculture and the environment.

### BRIEF DESCRIPTION OF FIGURES

FIG. 1 is a picture of maize growth (without treatment) after 6 eight weeks under different nitrogen fertilization (0 kgN/ha, 21 kgN/ha, 42 kgN/ha). Nutrient-poor Oxisol soil was used as test soil (substrate).

FIG. 2 is a picture of maize growth at 21 kgN/ha, once without (left) and once with application of Priestia aryabhattai DSM 34352, which was applied in a mixture with S301® to the leaf only.

FIG. 3 is a graph showing shoot dry matter of maize at different nitrogen fertilizer levels, with and without application of Priestia aryabhattai DSM 34352, which was applied in a mixture with S301®.

FIG. 4 is a graph showing root dry matter of maize at different nitrogen fertilizer levels, with and without application of Priestia aryabhattai DSM 34352, which was applied in a mixture with S301®.

FIG. 5 is a graph showing the increase in yield of the crop in the presence of Priestia aryabhattai DSM 34352.

### DESCRIPTION OF THE INVENTION

The present invention attempts to solve the problems above by providing a novel isolated endophyte from Priestia aryabhattai that is capable of being an effective and efficient biostimulant and/or biofertilizer. These newly isolated endophytes, which due to their unique genetic equipment and/or in the presence of specific adjuvants, are able to enable and especially enforce the penetration of these endophytes into the plant or part thereof to which the endophyte is brought into contact with. In particular, in the presence of the right adjuvant, the newly isolated Priestia aryabhattai strain is able to penetrate and proliferate in the plant tissue or seed. This way, (a) biotic stress can be reduced and thus the survivability or effectiveness of the organism in the plant is significantly increased. Inside the plant or seed or any part thereof, the active Priestia aryabhattai strain is in a regulated homeostasis, which increases both the efficacy and survivability of the nitrogen-fixing endophyte.

According to one aspect of the present invention, there is provided, a substantially purified or isolated endophyte, wherein the endophyte is a strain of Priestia aryabhattai which provides bioprotection and/or biostimulant phenotypes to plants into which it is introduced, wherein the strain of Priestia aryabhattai has Accession Number DSM 34352.

The newly isolated endophyte, Priestia aryabhattai, according to any aspect of the present invention solves the problem of low stability of microbial nitrogen fixers as it is an endophytic organism that has both an unprecedented high nitrogen fixation capacity and the ability to penetrate into the endosphere of the plant that most other existing endophytes used in agriculture do not have.

The endophyte according to any aspect of the present invention was isolated from the inner plant tissue of a copper flower (Minuartia verna subsp. hercynica) and typed and sequenced as Priestia aryabhattai sp. Greenhouse experiments demonstrated that Priestia aryabhattai is capable of providing significant amounts of enzymatically fixed nitrogen to the plant in both seed and foliar applications to maize (Zea mays, variant LG 31.224). Thus, maize inoculated or sprayed with Priestia aryabhattai was able to show the same or even improved growth as fully fertilized maize (42 kg/ha) when fertilized only with half of the synthetic nitrogen fertilizer (21 kg/ha). In comparison, significantly reduced growth was observed in maize not inoculated with Priestia aryabhattai at 21 kgN/ha.

As used herein, the term “endophyte” is an endosymbiont, which refers to a bacterial or fungal strain that lives within a plant for at least part of its life cycle without causing apparent disease. In particular, the bacteria or the fungus is closely associated with the plant where the term ‘closely associated’ refers to the bacteria or fungus living on, in or in close proximity to the plant. For example, it may be endophytic, and living within the internal tissues of the plant, or epiphytic, and growing externally on the plant. There are many different endophytes that have been discovered. However, only a few have been commercially used as endophytic inoculants for agriculture such as arbuscular mycorrhizae, rhizobia, and Azospirillium. Clavicipitaceous fungi is also an endophyte that is used in agriculture. The endophyte according to any aspect of the present invention is a strain of Priestia aryabhattai with Accession Number DSM 34352.

As used herein the term “substantially purified” refers to an endophyte being free of other organisms. The term includes, for example, an endophyte in axenic culture. Particularly, the endophyte is at least about 90% pure, more particularly at least about 95% pure, even more particularly at least about 98%, 99% or 99.5% pure.

As used herein the term ‘isolated’ refers to an endophyte according to any aspect of the present invention that is removed from its original environment (e.g., the natural environment if it is naturally occurring). For example, a naturally occurring endophyte present in a living plant is not isolated, but the same endophyte separated from some or all of the coexisting materials in the natural system, is isolated. In particular, the isolated endophyte according to any aspect of the present invention may be a pure culture of a single strain and this single strain was submitted to the German Collection of Microorganisms and Cell Cultures (DSMZ) located in Inhoffenstraße 7B, 38124 Braunschweig, Germany on 11 Aug. 2022 and has Accession Number DSM 34353. Particles for keeping and modifying cells are available from the prior art, for example Sambrook/Fritsch/Maniatis (1989).

As used herein the term “bioprotection and/or biostimulant” may refer to the endophyte according to any aspect of the present invention possessing genetic and/or metabolic characteristics that result in a beneficial phenotype in a plant harbouring, or otherwise associated with, the endophyte. Such beneficial properties or phenotypes resulting from the endophyte being present in the plant include improved resistance to pests and/or diseases, improved tolerance to water and/or nutrient stress, enhanced biotic stress tolerance, enhanced drought tolerance, enhanced water use efficiency, reduced toxicity and enhanced vigour in the plant with which the endophyte is associated, in comparison to a plant which is not associated with the endophyte according to any aspect of the present invention or to a endophyte such as standard toxic (ST) endophyte. In particular, the bioprotection and/or biostimulant phenotype according to any aspect of the present invention includes nitrogen fixation in the plant into which the endophyte is introduced.

The pests and/or diseases may include, but are not limited to, fungal and/or bacterial pathogens, particularly, fungal. In one example, the endophyte may result in the production of the bioprotectant compound in the plant with which it is associated.

As used herein, the term ‘bioprotectant compound’ refers to a compound that provides or aids in providing bioprotection to the plant with which it is associated against pests and/or diseases, such as bacterial and/or fungal pathogens. A bioprotectant compound may also be known as a ‘biocidal compound’.

As used herein, the term ‘biostimulant’ refers to any substance or microorganism applied to plants with the aim to enhance nutrition efficiency, abiotic stress tolerance and/or crop quality traits, regardless of its nutrients content. The endophyte according to any aspect of the present invention acts as a biostiumulant to the plant and/or part thereof to which it comes in contact with. A more detailed definition of biostimulant is provided at least in Ricci, M., General Principles to Justify Plant Biostimulant Claims, Frontiers in Plant Science (2019), 10.

As used herein the term ‘introduce’ refers to the contact and/or treatment of a plant or part thereof with an endophyte where the endophyte is delivered to the plant. In particular, the endophyte is introduced into the plant or part thereof to encourage the endophyte to grow there. Any method of introduction of the endophyte according to any aspect of the present invention to the plant or part thereof may be used. For example, the endophyte may be sprayed on or inoculated in the plant or part thereof. In particular, the endophyte may be inoculated for at least 1, 2, 3, 4, 5, 6, 7, 8, 9, 10, 11, 12, 13 or 14 days before they start to grow in the plant. In one example, the endophyte may be sprayed on the leaves of the plant (i.e. foliar application. In this example, the spraying process takes place either only in the early development stage of the plant (around the six-leaf stage) or multiple times (around the six-leaf stage and several stages of the emerging/adult plant). A skilled person would be capable of identifying the best introduction process to be used on the plant. In particular, the plant or part thereof may be infected with the endophyte by a method known in the art. More in particular, the plant or part thereof may be infected with the endophyte using a method selected from the group consisting of inoculation, spraying, breeding, crossing, hybridisation, transduction, transfection, transformation and/or gene targeting and combinations thereof.

In particular, the endophyte according to any aspect of the present invention is the Priestia aryabhattai strain with Accession Number DSM 34352. In particular, the sequence of the strain has been divided into loci and comprises the nucleotide sequence of SEQ ID NOs: 1-254 and variants thereof. In particular, the sequence of the 16S ribosomal RNA is SEQ ID NO:200.

The term “variant”, as used herein, comprises amino acid or nucleic acid sequences, respectively, that are at least 70, 75, 80, 85, 90, 92, 94, 95, 96, 97, 98 or 99% identical to the reference amino acid or nucleic acid sequence, wherein preferably amino acids other than those essential for the function, for example the catalytic activity of a protein, or the fold or structure of a molecule are deleted, substituted or replaced by insertions or essential amino acids are replaced in a conservative manner to the effect that the biological activity of the reference sequence or a molecule derived therefrom is preserved. The state of the art comprises algorithms that may be used to align two given nucleic acid or amino acid sequences and to calculate the degree of identity, see Arthur Lesk (2008), Thompson et al., 1994, and Katoh et al., 2005. The term “variant” is used synonymously and interchangeably with the term “homologue”. Such variants may be prepared by introducing deletions, insertions or substitutions in amino acid or nucleic acid sequences as well as fusions comprising such macromolecules or variants thereof. In one example, the term “variant”, with regard to amino acid sequence, comprises, in addition to the above sequence identity, amino acid sequences that comprise one or more conservative amino acid changes with respect to the respective reference or wild type sequence or comprises nucleic acid sequences encoding amino acid sequences that comprise one or more conservative amino acid changes. In one example, the term “variant” of an amino acid sequence or nucleic acid sequence comprises, in addition to the above degree of sequence identity, any active portion and/or fragment of the amino acid sequence or nucleic acid sequence, respectively, or any nucleic acid sequence encoding an active portion and/or fragment of an amino acid sequence. The term “active portion”, as used herein, refers to an amino acid sequence or a nucleic acid sequence, which is less than the full-length amino acid sequence or codes for less than the full-length amino acid sequence, respectively, wherein the amino acid sequence or the amino acid sequence encoded, respectively retains at least some of its essential biological activity.

According to another aspect of the present invention, there is provided a biostimulant comprising an isolated endophyte, wherein the endophyte is a strain of Priestia aryabhattai with Accession Number DSM 34352. The endophyte is according to any aspect of the present invention.

According to a further aspect of the present invention, there is provided a composition comprising:
an isolated endophyte, wherein the endophyte is a strain of Priestia aryabhattai, and
an adjuvant,
wherein the strain of Priestia aryabhattai has Accession Number DSM 34352.

The composition according to any aspect of the present invention solves the problem of low uptake rates and uptake kinetics of endophytic organisms into the endosphere of the plant. Adjuvants enable both an efficient uptake through stomata on the upper and most prominent on the lower surface of the plants, little injury wounds and growth gaps in the cuticula. The use of adjuvants to support the endophytic process (the penetration of the microorganism, particularly Priestia aryabhattai into the plant cell) allows the use of lower drug concentrations (CFU/mL or CFU/g) because uptake occurs more effectively than without the addition of adjuvants.

The endophyte is the Priestia aryabhattai with strain with Accession Number DSM 34352.

The isolated Priestia aryabhattai strain according to any aspect of the present invention has the appropriate genetic equipment to invade the plant tissue and survive and proliferate within the tissue. This in combination with the suitable penetration sites on the plant and/or seed surfaces with which the Priestia aryabhattai strain is inoculated enables the endophytes to successfully penetrate the plant tissue via the stomata of the leaves or via small injuries or growth gaps. Active penetration of the non-uniformly shaped cuticle has also been reported.

However, since both speed and kinetics of the endophytic process is limited in nature and thus occurs in most cases very slowly and host specific, the presence of an adjuvant in the composition according to any aspect of the present invention enables a successful uptake of the endophyte according to any aspect of the present invention into the plant tissue (endosphere) efficiently and effectively.

Usually when Priestia aryabhattai alone is applied to the soil, leaf, or seed, a slow endophytic process results in the exposure of the organism to biotic and abiotic stresses for a correspondingly long time. This can lead to a significant reduction in Priestia aryabhattai titer and thus reduced efficacy of the product. The presence of the adjuvant according to any aspect of the present invention cancels this negative effect and in fact increases the efficacy of the composition by accelerating the uptake of the organisms into the target plant or to enable it at all.

An ‘adjuvant’ as used herein, refers to an ingredient or a substance in the composition according to any aspect of the present invention that increases or modifies the activity of the other ingredients namely, the isolated Priestia aryabhattai strain. In particular, the adjuvants according to any aspect of the present invention are biocompatible adjuvant (active ingredient mediator) that are added to the suspension of microorganisms to form the composition according to any aspect of the present invention. Any adjuvant known in the art may be used in the composition according to any aspect of the present invention. In particular, the adjuvants according to any aspect of the present invention may be selected from the group consisting of (A), (B) or (C) and mixtures thereof wherein (A), (B) or (C) are:
(A) a polyglycerol ester with a general formula (I) of,

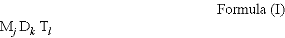

wherein, M is [C₃H₅(OR″)₂O_{1/2}],
   D is [C₃H₅(OR″)₁O_{2/2}],
   T is [C₃H₅O_{3/2}],
j=1 to 10, preferably 2 to 3, more preferably 2;
k=0 to 10, preferably greater than 0 to 5, more preferably 1 to 3;
l=0 to 3, preferably 0 to 1, more preferably 0;
wherein, the sum total of j+k+l is 1 to 20, preferably 2 to 4, more preferably 3
wherein the radicals R″ are each independently selected from the group consisting of acyl radicals R′—C(═O)— and H, with the proviso that at least one radical R″ is not equal to H;
wherein the radicals R′ are each independently selected from the group consisting of monovalent aliphatic, saturated or unsaturated hydrocarbon radicals with 3 to 39, preferably 7 to 21 more preferably with 9 to 17 carbon atoms;
(B) polyether-modified siloxanes of formula (II)

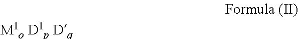

wherein, M¹ is P¹₃SiO_{1/2},
   D¹ is P¹₂SiO_{2/2}, D′ is P¹P²SiO_{2/2},
o is 2, p is between 0 and 0.1, q is between 1.0 and 1.15,
P¹ are independently hydrocarbyl having 1 to 8 carbon atoms,
P² are independently a polyether radical of the formula (III)

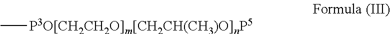

where
   m is from 3.4 to 11.0, n is from 2.5 to 8.0,
   and with the provisos that
   m/n is from 1.9 to 2.8,
   P³ are independently divalent hydrocarbyl radicals having 2 to 8 carbon atoms,
   P⁵ is hydrogen; and/or
(C) organomodified polysiloxane of formula (IV)

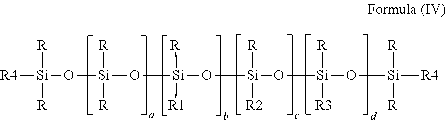

where a+b+c+d+2=20 to 210,
   a is 15 to 205, b is 1 to 12, c is 1 to 12, d is 1 to 12,
the R radicals are each independently identical or different, aliphatic or aromatic hydrocarbyl radicals having 1 to 10 carbon atoms,
the R4 radicals are each independently identical or different R, R1, R2 or R3 radicals,
the R1, R2 and R3 radicals are each independently different polyether radicals of general formula (V)

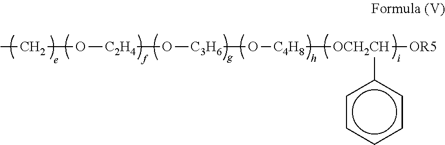

where
   e is 3 to 11, f is 6 to 30, g is 0 to 15, h is 0 to 5, i is 0 to 5, and
   R5 are independently the same or different and are each a methyl, acetyl or hydrogen radical.

More in particular, the adjuvants may be selected from the group consisting of BREAK-THRU® S 301, BREAK-THRU® SP 133, BREAK-THRU® S 255. The adjuvants used according to any aspect of the present invention leads to a reduction of the surface tension at the stomata or where there are injuries and thus to a lower rejection or an improved flow of the particles (microorganisms) through the orifices into the plant or part thereof where the microorganisms particularly Priestia aryabhattai with is inoculated. In particular, the use of adjuvants enables/accelerates the endophytic process of bacteria uptake. In addition, enhanced uptake also allows translocation of bacteria across the phloem from the leaf to the root, from where nitrogen fixation can also be enhanced. This results in the locally applied biostimulant acquiring a systemic character. Based on its systemic character, there is the advantage that in shoot-forming plants also the shoot is already inoculated with the biostimulant and thus passes the active ingredient on to the new generation.

The use of adjuvants also allows a uniform distribution of the Priestia aryabhattai from the upper leaf surface to the lower leaf surface where most (opened) stomata are present. Thus, faster and more widespread penetration of endophytes into plant tissue is enabled. Without the use of biocompatible adjuvants, especially in the case of foliar application, reaching the stomata on the underside of the leaf would be particularly hard if not impossible. The use of adjuvants with “anti-rinse-off” properties also increases the residence time on the upper surface of the leaf and thus promotes endophytic uptake into the plant tissue. The presence of the adjuvant reduces early wash-off of the endophytic nitrogen fixating organisms.

In one example, the adjuvant is (A):
(A) a polyglycerol ester with a general formula (I) of,

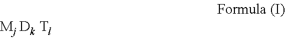

wherein, M is [C₃H₅(OR″)₂O_{1/2}],
   D is [C₃H₅(OR″)₁O_{2/2}],
   T is [C₃H₅O_{3/2}],
j=1 to 10, preferably 2 to 3, more preferably 2;
k=0 to 10, preferably greater than 0 to 5, more preferably 1 to 3;
l=0 to 3, preferably 0 to 1, more preferably 0;
wherein, the sum total of j+k+l is 1 to 20, preferably 2 to 4, more preferably 3
wherein the radicals R″ are each independently selected from the group consisting of acyl radicals R′—C(═O)— and H, with the proviso that at least one radical R″ is not equal to H;
wherein the radicals R′ are each independently selected from the group consisting of monovalent aliphatic, saturated or unsaturated hydrocarbon radicals with 3 to 39, preferably 7 to 21 more preferably with 9 to 17 carbon atoms.

In particular, at least one radical R″ corresponds to a radical of the formula R′—C(O)—.

More in particular, M, D and T may be:

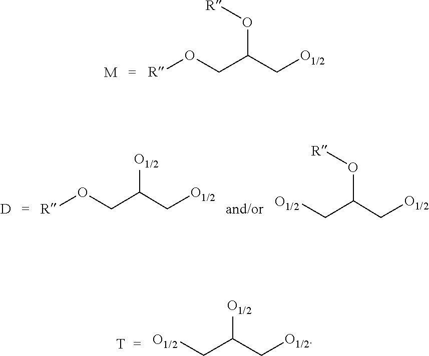

Even more in particular, the polyglycerol esters of the mixture according to any aspect of the present invention is of the Formula (I(a)):

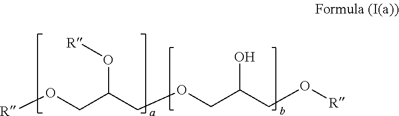

wherein
a=1 to 10, preferably 2 to 3, more in particular 2;
b=0 to 10, preferably greater than 0 to 5, more in particular 1 to 3;
with the proviso that:
a+b=2 to 20, preferably 2 to 4, especially 3;
wherein the radicals R″ are each independently selected from the group consisting of acyl radicals R′—C(═O)— and H, with the proviso that at least one radical R is not equal to H; where the radicals R′ are each independently selected from the group consisting of monovalent aliphatic, saturated or unsaturated hydrocarbon radicals with 3 to 39, preferably 7 to 21, in particular with 9 to 17 carbon atoms.

The polyglycerol esters of the composition according to any aspect of the present invention may have more than one radical R″ of the form R′—C(═O)—, particularly at least 2, more particularly at least 3.

The radicals R″ of the formula R′—C(O)— may be independent of each another identical or different acyl radicals of saturated or unsaturated fatty acids, where the fatty acids include 4 up to 40 carbon atoms, particularly, the fatty acids are selected from the group consisting of butyric acid (butanoic acid), caproic acid (hexanoic acid), caprylic acid (octanoic acid), capric acid (decanoic acid), lauric acid (dodecanoic acid), myristic acid (tetradecanoic acid), palmitic acid (hexadecanoic acid), stearic acid (octadecanoic acid), arachidic acid (eicosanoic acid), behenic acid (docosanoic acid), lignoceric acid (tetracosanoic acid), palmitoleic acid ((Z)-9-hexadecenoic acid), oleic acid ((Z)-9-hexadecenoic acid), elaidic acid ((E)-9-octadecenoic acid), cis-vaccenic acid ((Z)-11-octadecenoic acid), linoleic acid ((9Z,12Z)-9,12-octadecadienoic acid), alpha-linolenic acid ((9Z,12Z,15Z)-9,12,15-octadecatrienoic acid), gamma-linolenic acid ((6Z,9Z,12Z)-6,9,12-octadecatrienoic acid), di-homo-gamma-linolenic acid ((8Z,11Z,14Z)-8,11,14-eicosatrienoic acid), arachidonic acid ((5Z,8Z,11Z,14Z)-5,8,11,14-eicosatetraenoic acid), erucic acid ((Z)-13-docosenoic acid), nervonic acid ((Z)-15-tetracosenoic acid), ricinoleic acid, hydroxystearic acid, undecenoic acid, and mixtures thereof. In one example, the fatty acid may be a mixture of rapeseed oil acids, soya fatty acids, sunflower fatty acids, peanut fatty acids and tall oil fatty acids. In particular, for this context, the fatty acids may be radicals of oleic acid. When calculating the HLB value, the molar mass of the lipophilic molecule moiety is the arithmetic mean of the total of the molar masses of all of the radicals R′ which are present in the molecule.

Sources of suitable fatty acids or fatty acid esters, especially glycerides, can be vegetable or animal fats, oils or waxes. For example, lard, beef tallow, goose fat, duck fat, chicken fat, horse fat, whale oil, fish oil, palm oil, olive oil, avocado oil, seed kernel oils, coconut oil, palm kernel oil, cocoa butter, cottonseed oil, pumpkin seed oil, maize seed oil, sunflower oil, wheat germ oil, grape seed oil, soybean oil, peanut oil, lupine oil, rapeseed oil, mustard oil, castor oil, jatropa oil, walnut oil, jojoba oil, lecithin, for example based on soy, rapeseed, or sunflower, bone oil, claw oil, borage oil, lanolin, emu oil, deer tallow, marmot oil, mink oil, safflower oil, hemp oil, pumpkin oil, evening primrose oil, tall oil, as well as carnauba wax, beeswax, candelilla wax, ouricuri wax, sugar cane wax, retamow wax caranday wax, raffia wax, esparto wax, alfalfa wax, bamboo wax, hemp wax, Douglas fir wax, cork wax, sisal wax, flax wax, cotton wax, dammar wax, tea wax, coffee wax, rice wax, oleander wax or wool wax may be sources of fatty acids or fatty acid esters.

In particular, the polyglycerol ester compounds have the formulas (I), or (I(a)) with an arithmetic mean of 2.9 to 3.1 radicals of the form R′—C(═O)— and an HLB value of 4 to 6.5.

More in particular, the polyglycerol ester compounds have the formula (I(a)) with the sum a+b being 3 and the arithmetic mean 2.9 to 3.1 radicals of the form R′—C(═O)— and an HLB-value of 4 to 6.5.

Even more in particular, the polyglycerol ester compounds may be of the formula (I(a)) which have an arithmetic mean of 2.9 to 3.1 radicals of the form R′—C(═O)— and an HLB value of 4 to 6.5, where the acyl residues of fatty acid mixtures containing oleic acid, stearic acid, palmitic acid and gamma-linolenic acid, and said fatty acids particularly making up at least 85% by weight in the fatty acid mixture.

In one example, the polyglycerol ester compounds may be of the formula (I(a)) which have an arithmetic mean of 2.9 to 3.1 radicals of the form R′—C(═O)— and an HLB value of 4 to 6.5, the acyl residues originate from fatty acid mixtures containing oleic acid, stearic acid, palmitic acid and gamma-linolenic acid, and said fatty acids particularly make up at least 85% by weight in the fatty acid mixture.

In another example, the polyglycerol ester compounds used according to any aspect of the present invention may be of the formula (I(a)) which have an arithmetic mean of 2.9 to 3.1 radicals of the form R′—C(═O)— and an HLB value of 4 to 6.5, the mass fraction of oleic acyl residues is at least 75%, particularly 85%, more particularly 95% based on the mass of all acyl residues. Even more in particular, the polyglycerol ester is triglycerol trioleate.

A more thorough disclosure of the adjuvant (B) is provided at least in U.S. Pat. No. 10,390,530B2.

In one example, the adjuvant is (B):
(B) polyether-modified siloxanes of formula (II)

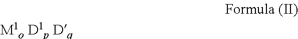

wherein, M¹ is P¹₃SiO_{1/2},
   D¹ is P¹₂SiO_{2/2}, D′ is P¹P²SiO_{2/2},
is 2,
p is between 0 and 0.1, particularly 0
q is between 1.0 and 1.15, particularly between 1.0 and 1.10, especially particularly between 1.00 and 1.05,
P¹ are independently hydrocarbyl having 1 to 8 carbon atoms, particularly methyl, ethyl, propyl or phenyl radicals, especially particularly methyl radicals,
P² are independently a polyether radical of the formula (III)

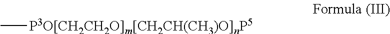

where
m is from 3.4 to 11.0, particularly 3.6 to 9.9, more particularly 4.5 to 8.5,
n is from 2.5 to 8.0, particularly 2.7 to 7.5, more particularly 3.0 to 6.0,
and with the provisos that
m/n is from 1.9 to 2.8,
P³ are independently divalent hydrocarbyl radicals having 2 to 8 carbon atoms, particularly ethylene, propylene, 1-methylpropylene, 1,1-dimethylpropylene radical, especially particularly —CH2CH2CH2—,
P⁵ is hydrogen.

The polyether-modified siloxanes of formula (II) have a biodegradability of greater than 60%, more particularly of greater than or equal to 63% and especially particularly of greater than or equal to 65%, the maximum value being 100%.

In particular, the polyether radical, calculated without P³O and calculated without P⁵, has a molar mass M(PE) calculated by 44 g/mol*m+58 g/mol*n where the indices m and n relate to formula (III). More in particular, the values of M(PE) are: lower limits M(PE) greater than 520 g/mol, particularly greater than 530 g/mol, more particularly greater than 535 g/mol; upper limit M(PE) less than 660 g/mol, particularly less than 630 g/mol, more particularly less than 600 g/mol. Even more in particular, the value of M(PE) is greater than 520 g/mol and less than 660 g/mol, especially greater than 535 g/mol and less than 600 g/mol. In particular, the sum total of m+n is greater than 9 up to 19, more in particular, than 9.5 up to 15 and even more in particular greater than 10 up to 12.

In one example, the polyether-modified siloxane used in the composition according to any aspect of the present invention is a polyether-modified siloxane of the formula (II) with an index c between 1 and 1.05, where the indices of the polyether radical of formula (III) are m from 3.4 to 11.0 and n from 2.5 to 8.0. In particular, the polyether-modified siloxane used in the composition according to any aspect of the present invention is a polyether-modified siloxane of the formula (II) with an index c between 1 and 1.05, where the ratio m/n is 1.9 to 2.8. More in particular, the polyether-modified siloxane used in the composition according to any aspect of the present invention is a polyether-modified siloxane of the formula (II) with an index c between 1 and 1.05, where the molar mass of the polyether residue M(PE) is greater than 520 g/mol and less than 660 g/mol. Even more in particular, the polyether-modified siloxane used in the composition according to any aspect of the present invention is a polyether-modified siloxane of the formula (II) with an index c between 1 and 1.05, where the P⁵ radical is hydrogen or with an index c between 1 and 1.05, where the molar mass of the polyether residue M(PE) is greater than 520 g/mol and less than 660 g/mol and the P⁵ radical is hydrogen. In particular, the polyether-modified siloxane used in the composition according to any aspect of the present invention is a polyether-modified siloxane of the formula (II) does not include any further polyether-modified siloxanes apart from those of formula (II).

A more thorough disclosure of the adjuvant (B) is provided at least in U.S. Pat. No. 10,299,471B2.

In one example, the adjuvant is (C), an organomodified polysiloxane of formula (IV)

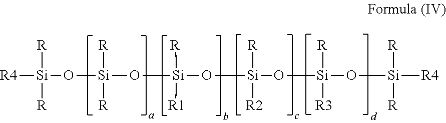

where a+b+c+d+2=20 to 210, preferably 30 to 100, especially 40-60,
a is 15 to 205, preferably 35 to 45,
b is 1 to 12, preferably 1 to 8, especially 2 to 6,
c is 1 to 12, preferably 1 to 8, especially 2 to 6,
d is 1 to 12, preferably 1 to 8, especially 2 to 6,
the R radicals are each independently identical or different, aliphatic or aromatic hydrocarbyl radicals having 1 to 10 carbon atoms, preferably methyl radicals,
the R4 radicals are each independently identical or different R, R1, R2 or R3 radicals,
the R1, R2 and R3 radicals are each independently different polyether radicals of general formula (V)

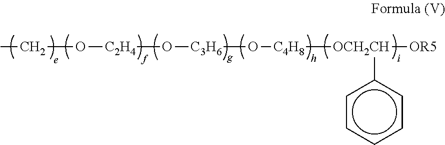

where
e is 3 to 11, preferably 3,
f is 6 to 30, preferably 10 to 30,
g is 0 to 15, preferably 0 to 10,
h is 0 to 5,
i is 0 to 5, and

R5 are independently the same or different and are each a methyl, acetyl or hydrogen radical preferably with the proviso that the molecular weight of the polyether radical of Formula (V) is greater than 200 g/mol, preferably from greater than 400 to 2000 g/mol, and the proportion of ethylene oxide is greater than 45% by mass in the polyether, and the percentage by mass of ethylene oxide in the polyether radical R2 is at least 9% by mass greater than the percentage of ethylene oxide in the polyether radical R1, based in each case on the polyether radicals of the Formula (V), where the radicals of the Formula (V) may each be formed randomly, in a gradient or in blocks.

In formula (V), the units designated by the index ‘g’ are those which have originated from propylene oxide, the units designated by the index ‘h’ are those which have originated from butylene oxide, and the units designated by the index ‘i’ are those which have originated from styrene oxide.

The indices ‘a to d’ and ‘e to i’ may be natural whole numbers, or weight averages. The indices are preferably weight averages.

A more thorough disclosure of the adjuvant (C) is provided at least in U.S. Pat. No. 8,580,225B2.

The composition according to any aspect of the present invention may comprise any one of the adjuvants (A), (B) or (C). In one example, the composition may comprise a mixture of adjuvants such as (A) and (B), (A) and (C), (B) and (C) or (A), (B) and (C). In another example, the composition according to any aspect of the present invention may comprise more than one adjuvant (A) or more than one adjuvant (B) or more than one adjuvant (C).

The composition according to any aspect of the present invention may further comprise an emulsifier. The emulsifier in the mixture according to any aspect of the present invention may be different from the adjuvant. The emulsifier may be selected from the group consisting of fatty acid esters of polyhydric alcohols and their polyalkylene glycol derivatives, polyglycol derivatives of fatty acids and fatty alcohols, sorbitan fatty acid esters, ethoxylated and/or propoxylated sorbitan fatty acid esters, propoxylated sorbitan fatty acid esters, alkylphenol ethoxylates, propoxylates, alkylphenol ethoxylates, aminoxylated oxides, amine oxides, propoxylated amine oxides, aminoxylated amine oxides, aminoxylated propylene oxides, acetylenediol surfactants, ethoxylated and/or propoxylated acetylenediols, silicone surfactants and mixtures thereof. In particular, the emulsifier is selected from the group consisting of sorbitan fatty acid esters and ethoxylated sorbitan fatty acid esters. More in particular, the emulsifier is an ethoxylated sorbitan fatty acid ester or mixtures thereof.

The acyloxy radicals of the sorbitan fatty acid ester or ethoxylated sorbitan fatty acid ester have 4 to 40, in particular 8 to 22, more in particular 10 to 18 carbon atoms and/or that the sorbitan fatty acid ester or ethoxylated sorbitan fatty acid ester has 0 to 40, particularly 10 to 30, more particularly 15 to 25 oxyethylene groups. The fatty acids or fatty acid residues of the sorbitan fatty acid esters are particularly defined like the fatty acids or fatty acid residues of the polyglycerol esters. The acyl radicals (also referred to as alkanoyl radicals) are particularly derived from fatty acid mixtures containing oleic acid, stearic acid, palmitic acid and gamma-linolenic acid, said fatty acids particularly making up at least 85% by weight in the fatty acid mixture. Ethoxylated sorbitan fatty acid esters are particularly used, the mass fraction of oleic acid acyl residues being at least 75%, particularly 85%, more particularly 95%, based on the mass of all acyl residues.

The emulsifier according to any aspect of the present invention has an HLB value of greater than or equal to 9, particularly greater than or equal to 10, more particularly greater than or equal to 11. The HLB value may be a maximum of 16, more particularly a maximum of 15, even more particularly a maximum of 13. In particular, the emulsifier has an HLB value of 9 to 16, particularly 10 to 15, more particularly 11 to 13. The HLB value is determined as described above. The HLB value of the sorbitan fatty acid esters and/or ethoxylated sorbitan fatty acid esters is particularly determined as for the polyglycerol esters. The molar mass of the lipophilic part of the molecule results from the arithmetic mean of the sum of the molar masses of all radicals R′″ present in the molecule as part of the acyl radicals R′″—(CO)—. The radicals R′″ are preferably as defined for the polyglycerol esters. The radical R′″ as part of an acyl radical R′″—(CO)— of the sorbitan fatty acid ester or ethoxylated sorbitan fatty acid ester is particularly selected from the group consisting of monovalent aliphatic, saturated or unsaturated hydrocarbon radicals with 3 to 39, preferably 7 to 21, particularly 9 to 17 carbon atoms. The calculation of the molar mass of the entire molecule is carried out as defined above. In particular, the emulsifier is polyethylene glycol-20-sorbitan trioleate. The number 20 indicates the average number of ethylene oxide units in the polyethylene glycol residue.

The HLB value of the polyglycerol ester and of the emulsifier are matched to one another. The polyglycerol ester has an HLB value of less than or equal to 8, particularly less than or equal to 7, more particularly less than or equal to 6.5, and the at least one emulsifier has an HLB value of greater than or equal to 9, particularly greater than or equal to 10, in particular greater than or equal to 11. The at least one polyglycerol ester has an HLB value of 0.5 to 8, particularly from 1 to 7, more particularly from 2 to 6.5 and the at least one emulsifier has an HLB value of 9 to 16, particularly from 10 to 15, more particularly from 11 to 13. In particular, the emulsifier is polyethylene glycol 20 sorbitan trioleate.

According to a further aspect of the present invention, there is provided a composition comprising a culture medium of an isolated endophyte, and the endophyte, wherein the endophyte is a strain of Priestia aryabhattai with Accession Number DSM 34352.

The strains and compositions according to any aspect of the present invention can be obtained by culturing the strains of Priestia aryabhattai according to methods well known in the art, including by using the appropriate media. Conventional large-scale microbial culture processes include submerged fermentation, solid state fermentation, or liquid surface culture. The endophyte may be cultured under aerobic or anaerobic conditions and may be cultured in a bioreactor. The endophytes, and metabolites in culture media resulting from culturing may be used directly or concentrated by conventional industrial methods, such as centrifugation, tangential-flow filtration, depth filtration, and evaporation. The concentrated fermentation broth may be washed, for example via a diafiltration process, to remove residual fermentation broth and metabolites.

The fermentation broth or broth concentrate can be dried with or without the addition of carriers using conventional drying processes or methods such as spray drying, freeze drying, tray drying, fluidized-bed drying, drum drying, or evaporation. The resulting dry products may be further processed, such as by milling or granulation, to achieve a specific particle size or physical format. Carriers may also be added post-drying. In particular, the preparation of the strains is a supernatant of the fermentation broth. In one example, the composition according to any aspect of the present invention may be prepared according to the method provided in EP21198571 or EP21202623 wherein the endophyte according to any aspect of the present invention is first spray-dried and then brought into contact with at least one adjuvant. In particular, the adjuvant may be (A), (B) or (C) according to any aspect of the present invention. More in particular, the adjuvant is (A), a polyglycerol ester with general formula (I). Even more in particular, the polyglycerol ester is combined with at least one emulsifier according to any aspect of the present invention.

According to a further aspect of the present invention, there is provided a plant or part thereof infected with one or more endophytes according to any aspect of the present invention. In particular, the plant or part thereof infected with the endophyte may produce a bioprotectant compound. In particular, the plant or part thereof includes an endophyte-free host plant or part thereof stably infected with said endophyte.

Any plant or part thereof may be inoculated with the endophyte according to any aspect of the present invention. In particular, the plant inoculated with the endophyte may be a grass or non-grass plant suitable for agriculture, specifically a forage, turf, or bioenergy grass, or a grain crop or industrial crop. More in particular, the grain crop or industrial crop species may be selected from the group consisting of wheat, barley, oats, corn/maize, any grain legumes such as chickpeas, triticale, fava beans, lupins, field peas, canola, cereal rye, vetch, lentils, millet/panicum, safflower, linseed, sorghum, sunflower, maize, canola, mungbeans, soybeans, oilseed crops, tomato and cotton.

The endophyte according to any aspect of the present invention may be transferred through seed from one plant generation to the next. The endophyte may then spread or locate to other tissues as the plant grows, i.e., to roots. Alternatively, or in addition, the endophyte may be recruited to the plant root, e.g. from soil, and spread or locate to other tissues. According to a further aspect of the present invention, there is provided a plant, plant seed or other plant part derived from a plant or part thereof according to any aspect of the present invention. The plant, plant seed or other plant part may produce a bioprotectant compound.

The endophyte-infected plant or part thereof may be cultivated by known techniques. The person skilled in the art may readily determine appropriate conditions depending on the plant or part thereof to be cultivated.

According to another aspect of the present invention, there is provided a method of producing a composition for bioprotection and/or biostimulant, the method comprising combining:
an isolated endophyte, wherein the endophyte is a strain of Priestia aryabhattai, and
an adjuvant,
wherein the strain of Priestia aryabhattai with Accession Number DSM 34352.

According to yet another aspect of the present invention, there is provided a method of providing bioprotection to a plant or part thereof, the method comprising contacting the composition according to any aspect of the present invention to the plant or part thereof.

The part thereof of the plant may be, for example, a seed. In one example, the composition according to any aspect of the present invention is introduced to the leaf, roots or the seeds of the plant. In particular, the composition according to any aspect of the present invention is introduced to the leaf of the plant. Applying the endophytes to the foliage allows the user to treat the growing or adult plant at several distinct times after sowing (if needed). In contrast, only a single application is possible during soil application (prior to seeding) or seed application (during seed treatment). Further, foliar application of Priestia aryabhattai allows for reduced use of the microbial agent because higher CFU loss (several log levels) can be expected within a short period of time (lower stability of Gram-negative organisms on the seed or in the soil) when the seed or soil is inoculated. According to yet another aspect of the present invention, there is provided a use of the composition according to any aspect of the present invention to provide bioprotection to a plant or part thereof.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

### Strain Collection

For the collection of Priestia aryabhattai strain with Accession Number DSM 34352, different plants were collected from different sites. The sites were selected based on their distinctive habitats. A list of type of sites and plants that were used to collect Priestia aryabhattai DSM 34352 is provided in Table 1.

TABLE 1
List of sites and plants used to collect
Priestia aryabhattai DSM 34352

| sites | plant (crop) species |
|---|---|
| heavy metal vegetation | Minuartia verna, |
| | Armeria maritima |
| | subsp. Halleri, |
| | Silene vulgaris |
| inland sand dunes | Festuca ovina |
| Elbe floodplains, unfertilized for 20 years | mixed vegetation |
| chernozem (fertile loess area) | wheat, maize |
| Luvisol (fertile Loess area) | wheat |
| Elbe floodplains, unfertilized for 20 years | mixed vegetation |
| “Gäuboden” (fertile loess area) | wheat, maize |

A trowel or small shovel was used to gently excavate the soil around individual plants, and to lift the roots with minimal disturbance. The removal of root parts was carried out gently and without damaging the entire root system. Similarly, leaf material was harvested from individual plants without causing damage to the plants. Plant material was placed into separate plastic bag, and the bags were then carefully packed into insulated and chilled transport bags. After field collection, plant material was chilled in a refrigerator at 5° C. till further use.

The leaves and roots of each plant were washed separately under slow running tap water for 15 minutes in sterile distilled water to remove adhering soil particles and the majority of microbial surface epiphytes as a part of pre-treatment. Then the samples were rinsed three times for one minute each time with sterile distilled water in the laminar air flow cabinet.

Roots and leaves were cut into sections. The plant material was put in petri dishes, soaked in distilled water and drained. It was rinsed in 70% ethanol for 30 seconds and then sterilized with 3% sodium hypochlorite for 3 minutes for roots and for 5 minutes for leaves. The tissue was then washed ten times with sterile water. Validation of the surface sterilization procedure was done by culturing aliquots of water from the last rinsing onto nutrient media.

After proper drying of surface sterilized plant material in the laminar air flow cabinet, roots and leaves were cut into pieces 2-3 cm long. The external portion of the leaves approximately 0.5 cm from the margin was removed with a sterile blade. Each piece of both roots and leaves were placed on nutrient agar medium and supplemented with 100 mg L-1 of cycloheximide to inhibit possible fungal development. Plates with plant tissues were sealed using parafilm tape and incubated at 28±2° C. in order to recover the maximum possible colonies of bacterial endophytes. After 48 hours, morphologically different bacterial colonies were selected from root tips and leaf segments and after fourfold serial dilution repeatedly streaked in order to achieve enriched bacterial isolates. In order to obtain pure cultures enriched cultures were streaked out on individual 50% TS agar plates and incubated for 2-3 days at 30° C.

Colony morphology was investigated by eye to examine purity of the cultures. Colonies that appeared different were isolated and again streaked on separate 50% TS agar plates for a new cultivation. That way, mixed cultures were divided into monoseptic isolates. The pureness was confirmed by microscopic analysis and subsequent alignments of 16S sequences with corresponding sequences from the NCBI rRNA/ITS databases using “blastn”. The latter procedure was provided as a service by VERMICON (Hallbergmoos, Germany).

Cell material from these re-plate cultures were resuspended in 50% glycerol solution as done with the primary cell material and stored at −80° C. To test re-vitalizing, new cultures were inoculated from these cryo-stocks. A growing culture was labelled “pure” and “cryo-saved”. For genome sequencing, strains of particular interest were grown in liquid culture, spun down and resuspended in 70% ethanol. About 1×10⁸ cells of those strains were sent to LGC Biomics (Berlin, Germany) for whole genome sequencing. It was confirmed to be a novel strain. This strain was Priestia aryabhattai DSM 34352.

### Example 2

### Efficacy Analysis

To determine the efficacy of nitrogen fixation of Priestia aryabhattai DSM 34352 as an endophytic biostimulant, untreated maize seeds (Zea mays, variant LG 31.224) were planted in poor sandy soil (Oxisol) with little organic matter (important that N-content is low, so that the effect of free-living N fixing bacteria becomes visible).

Five seeds were planted per pot which further have been thinned out to three plants after germination in order to avoid minor number of replicates. Fertilizer dose rate was calculated on basis of 1,000,000 kg soil/ha. Fertilization was conducted with phosphate, potassium and magnesium sulfate (25 kg P/ha, 50 kg K/ha and 15 kg Mg/ha, respectively). Fertilization with Nitrogen was performed with DEMOGRAN® 45 (Domo, Germany) that contains water soluble 21% N and 24% S; (24 and 48 kg N/ha). 2 L pots were used and filled with 2 kg of sandy soil.

At leaf stage six (about 2 weeks after planting) Priestia aryabhattai DSM 34352 was applied to the leaves in combination with an adjuvant. In the process, 1 mL of spray volume was applied to the leaves of the young maize plant. The mixture contained 300.000 CFU of Paenibacillus xylanexedens DSM34353 and 0.1% of S301® (Evonik Industries, Germany) as adjuvant for surface wetting as well as stomata flooding. S301® has the formula of adjuvant (B) according to any aspect of the present invention. In particular, S301® has the formula

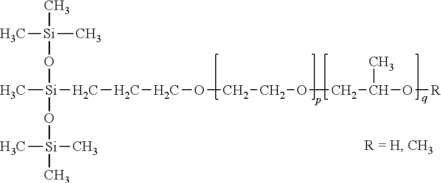

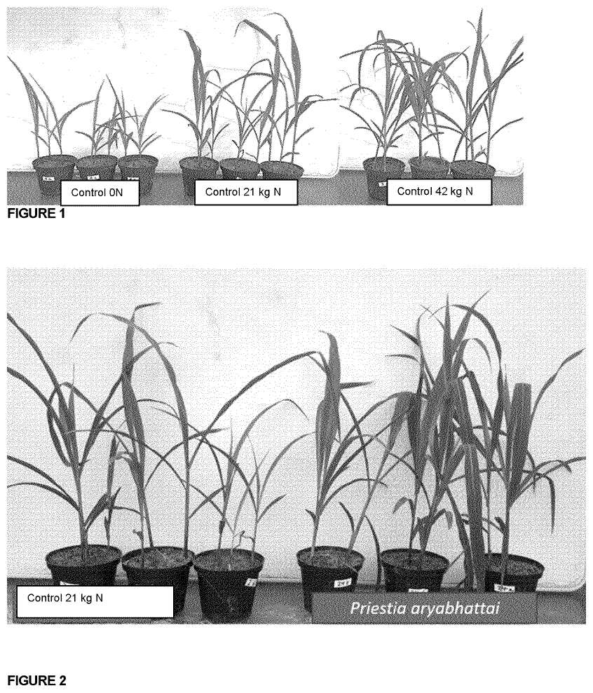

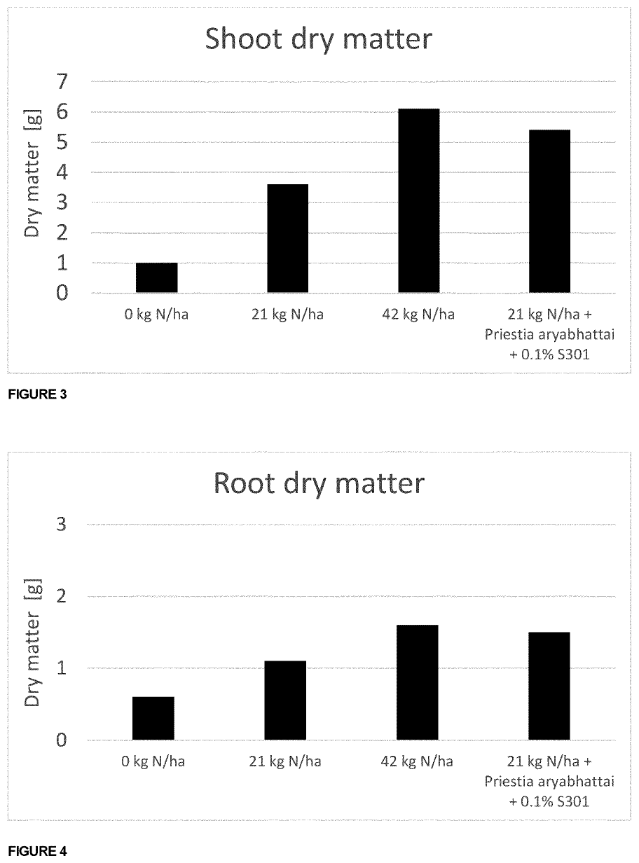

1 ml spray to the leaves of the pot corresponded to about 400 L spray per ha which resembled the standard application rate for field application. Maize plants were grown at temperatures between 2° and 25° C. with regular irrigation. Eight weeks after treatment (8 WAT) pictures were recorded (FIGS. 1 and 2) and dry mass of roots (FIG. 4) and shoots (FIG. 3) were determined.

It was shown that the efficacy of Priestia aryabhattai DSM 34352 in combination with adjuvant S301® was good.

Further, compounds with other microorganisms such as a biofertilizer Blue-N(Corteva, Inc, USA) with Methylobacterium symbioticum, Kreotec (Biofa) with Bacillus velezensis, Azospirillum brasilense and Herbaspirillum seropedicae, biofertilizer Utrisha™-N(Corteva, Inc, USA) with Methylobacterium symbioticum SB23, Blue-N(Corteva, Inc, USA) with Methylobacterium symbioticum, RhizoFert® (AbiTep, Germany) with Bacillus atrophaeus and RhizoFos with Pseudomonas fluorescens (Rizobacter, USA) were used for comparative studies as controls. These comparative products also contained microorganisms that were able to fix atmospheric nitrogen and were also applied to the leaf. However, the comparative products showed no or a lower growth-promoting effect with reduced anthropogenic nitrogen fertilization (21 kgN/ha).

These comparative trials showed that the use of Priestia aryabhattai DSM 34352 in combination with the adjuvant led to a better growth of the plant under N-limited conditions than when using Utrisha™-N, Blue-N or Kreotec.

### Example 3

Investigate the Effect of Different Mineral N-Fertilizing Doses on Efficacy of N-Fixing Bacterial Endophytes Priestia aryabhattai DSM 34352

The general objective of example 3 was to find out how much N the bacterial endophyte Priestia aryabhattai DSM 34352 can fix under field conditions (i.e. at different mineral fertilizing doses for crop selectivity and on yields (grain & shoots) under field conditions). When seed was treated, bacterial endophytes by wet seed treatment method was carried out. When foliar was applied, bacterial endophytes at 5-6 leaf stage was applied.

### Corn Seed Treatment

100,000 grains are planted, 1,000,000 CFU (bacterial units) are applied per seed. At 100,000 grains per ha: 1×10¹¹ CFU/ha

The individual bacterial suspensions each have 2.5×10⁸ CFU/mL, so 400 mL of experimental product is required per 100 000 grains. This results in 4 mL of experimental product (with 2.5×10⁸ CFU) per 1000 grains. A mix of 4 mL of experimental product with 1 mL of PREMAX and pickle to 1000 corn kernels was made, then left to dry in the dark (e.g. open paper bag). Nothing was added for the controls.

### Foliar Treatment

50,000 CFU (units) of experimental bacteria was added per plant. The plants were in the 5-6 leaf stage, a stage in which only about 10% or less of the area of the soil was covered with plants. With 100,000 plants/ha and 50,000 CFU, it resulted in a total of 5×10⁹ CFU/10% area of one ha—i.e. on 1 ha in 5×10¹⁰ CFU/ha. 200 mL/ha of experimental bacterial products with 2.5×10⁸ CFU/mL was sprayed—this resulted in 5×10¹⁰ CFU/ha. 10 mL of experimental product (2.5×10⁸ CFU/mL) was diluted with 90 mL of demineralized water—this yielded 100 mL of 2.5×10⁷ CFU/mL. Of these 100 mL (with 2.5×10⁷ CFU/mL), 20 mL/100 m2 was applied—or 2000 mL/ha. The Water application rate (spray liquid) was 300 L/ha and BREAK-THRU® SP 133:300 mL/ha (=0.1%) was added. All experiments were carried out in Germany.

The following experiments were carried out:

| | | | | Application | |
|---|---|---|---|---|---|
| No. | Product | N-Fertilization | | Technique | Dose |
| 1 | Control | 0 | N/ha | untreated | untreated |
| 2 | Priestia | 0 | N/ha | Seed treatment + | 1 × 10⁶ CFU |
| | aryabhattai | | | foliar application | per seed/50,000 |
| | | | | (1×) 5-6 leaf | CFU/plant |
| 3 | Control | 90 | kg N/ha | stage untreated | untreated |
| 4 | Priestia | 90 | kg N/ha | Seed treatment + | 1 × 10⁶ CFU |
| | aryabhattai | | | foliar application | per seed/50,000 |
| | | | | (1×) 5-6 leaf | CFU/plant |
| 5 | Control | 135 | kg N/ha | stage,untreated | untreated |
| 6 | Priestia | 135 | kg N/ha | Seed treatment + | 1 × 10⁶ CFU |
| | aryabhattai | | | foliar application | per seed/50,000 |
| | | | | (1×) 5-6 leaf | CFU/plant |
| 7 | Control | 180 | kg N/ha | stage untreated | untreated |

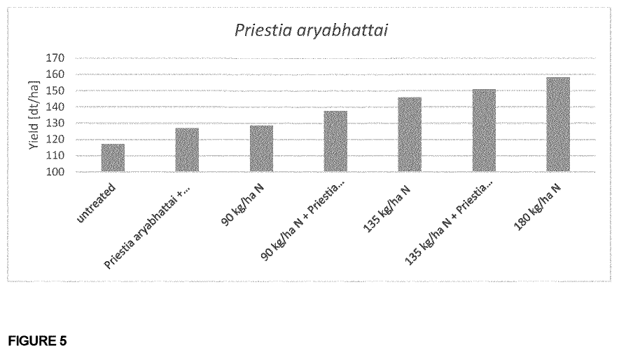

The results are provided below in Table 2 and FIG. 5.

TABLE 2
Results of Example 3

| No. | Experiment | Yield [dt/ha] |
|---|---|---|
| 1 | untreated | 117.2 |
| 2 | Priestia aryabhattai + | 127.0 |
| | BREAK-THRU ® SP133 | |
| 3 | 90 kg/ha N | 128.5 |
| 4 | 90 kg/ha N + Priestia aryabhattai + | 137.4 |
| | BREAK-THRU ® SP133 | |
| 5 | 135 kg/ha N | 145.9 |
| 6 | 135 kg/ha N + Priestia aryabhattai + | 150.9 |
| | BREAK-THRU ® SP133 | |
| 7 | 180 kg/ha N | 158.2 |

The results show, that total yield increases, when the biostimulant (i.e. Priestia aryabhattai) was applied in addition to the synthetic fertilizer.

1. A composition comprising:
an isolated endophyte, wherein the endophyte is a strain of Priestia aryabhattai, and
an adjuvant, wherein the Priestia aryabhattai strain has Accession Number DSM 34352.

2. The composition according to claim 1, wherein the adjuvant is selected from the group consisting of (A), (B) and (C):
(A) a polyglycerol ester with a general formula (I) of, 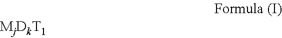
wherein, Mis [C₃H₅(OR″)₂O_{1/2}],
D is [C₃H₅(OR″)₁O_{2/2}],
T is [C₃H₅O_{3/2}],
j=1 to 10, preferably 2 to 3, more preferably 2;
k=0 to 10, preferably greater than 0 to 5, more preferably 1 to 3;
l=0 to 3, preferably 0 to 1, more preferably 0;
wherein, the sum total of j+k+l is 1 to 20, preferably 2 to 4, more preferably 3
wherein the radicals R″ are each independently selected from the group consisting of acyl radicals R′—C(═O)— and H, with the proviso that at least one radical R″ is not equal to H;
wherein the radicals R′ are each independently selected from the group consisting of monovalent aliphatic, saturated or unsaturated hydrocarbon radicals with 3 to 39, preferably 7 to 21 more preferably with 9 to 17 carbon atoms;
(B) polyether-modified siloxanes of formula (II) 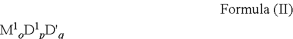
wherein, M¹ is P¹₃SiO_{1/2},
D¹ is P¹₂SiO_{2/2}, D′ is P¹P²SiO_{2/2},
o is 2, p is between 0 and 0.1, q is between 1.0 and 1.15,
P¹ are independently hydrocarbyl having 1 to 8 carbon atoms,
P² are independently a polyether radical of the formula (III) 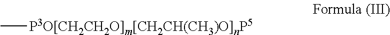
where
m is from 3.4 to 11.0, n is from 2.5 to 8.0,
and with the provisos that
m/n is from 1.9 to 2.8,
P³ are independently divalent hydrocarbyl radicals having 2 to 8 carbon atoms,
P⁵ is hydrogen; and/or
(C) organomodified polysiloxane of formula (IV) 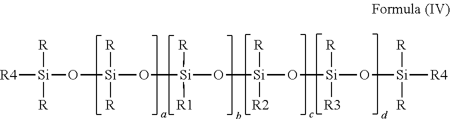
where a+b+c+d+2=20 to 210,
a is 15 to 205, b is 1 to 12, c is l to 12, dis 1 to 12,
the R radicals are each independently identical or different, aliphatic or aromatic hydrocarbyl radicals having 1 to 10 carbon atoms,
the R4 radicals are each independently identical or different R, R1, R2 or R3 radicals,
the R1, R2 and R3 radicals are each independently different polyether radicals of general formula (V) 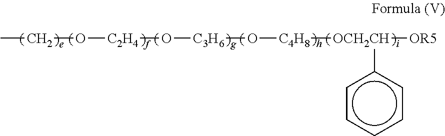
where
e is 3 to 11, f is 6 to 30, g is 0 to 15, h is 0 to 5, i is 0 to 5, and
R5 are independently the same or different and are each a methyl, acetyl or hydrogen radical.

3. The composition according to either claim 1, further comprising at least one emulsifier.

4. The composition according to claim 3, wherein the emulsifier is selected from the group consisting of sorbitan fatty acid esters, ethoxylated sorbitan fatty acid esters and mixtures thereof.

5. A substantially purified or isolated endophyte, wherein the endophyte is a strain of Priestia aryabhattai which provides bioprotection and/or biostimulant phenotypes to plants into which the endophyte is introduced, wherein the endophyte Priestia aryabhattai strain has Accession Number DSM 34352.

6. The endophyte according to claim 5, wherein the bioprotection and/or biostimulant phenotype includes nitrogen fixation in the plant into which the endophyte is introduced.

7. A biostimulant comprising an isolated endophyte, wherein the endophyte is a strain of Priestia aryabhattai with Accession Number DSM 34352.

8. The biostimulant according to claim 7, further comprising at least one adjuvant selected from the group consisting of (A), (B) and (C):
(A) a polyglycerol ester with a general formula (I) of, 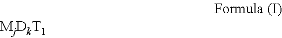
wherein, M is [C₃H₅(OR″)₂O_{1/2}],
D is [C₃H₅(OR″)₁O_{2/2}],
T is [C₃H₅O_{3/2}],
j=1 to 10, preferably 2 to 3, more preferably 2;
k=0 to 10, preferably greater than 0 to 5, more preferably 1 to 3;
l=0 to 3, preferably 0 to 1, more preferably 0;
wherein, the sum total of j+k+l is 1 to 20, preferably 2 to 4, more preferably 3
wherein the radicals R″ are each independently selected from the group consisting of acyl radicals R′—C(═O)— and H, with the proviso that at least one radical R″ is not equal to H;
wherein the radicals R′ are each independently selected from the group consisting of monovalent aliphatic, saturated or unsaturated hydrocarbon radicals with 3 to 39, preferably 7 to 21 more preferably with 9 to 17 carbon atoms;
(B) polyether-modified siloxanes of formula (II) 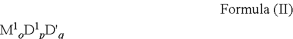
wherein, M¹ is P¹₃SiO_{1/2},
D′ is P¹₂SiO_{2/2}, D′ is P¹P²SiO_{2/2},
o is 2, p is between 0 and 0.1, q is between 1.0 and 1.15,
P¹ are independently hydrocarbyl having 1 to 8 carbon atoms,
P² are independently a polyether radical of the formula (III) 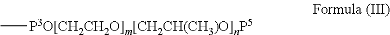
where
m is from 3.4 to 11.0, n is from 2.5 to 8.0,
and with the provisos that
m/n is from 1.9 to 2.8,
P³ are independently divalent hydrocarbyl radicals having 2 to 8 carbon atoms,
P⁵ is hydrogen; and/or
(C) organomodified polysiloxane of formula (IV) 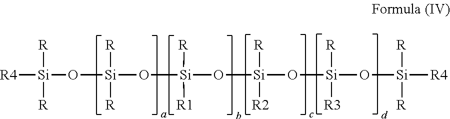
where a+b+c+d+2=20 to 210,
a is 15 to 205, b is 1 to 12, c is 1 to 12, dis 1 to 12,
the R radicals are each independently identical or different, aliphatic or aromatic hydrocarbyl radicals having 1 to 10 carbon atoms,
the R4 radicals are each independently identical or different R, R1, R2 or R3 radicals,
the R1, R2 and R3 radicals are each independently different polyether radicals of general formula (V) 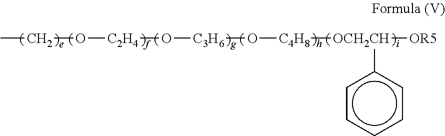
where
e is 3 to 11, f is 6 to 30, g is 0 to 15, h is 0 to 5, i is 0 to 5, and
R5 are independently the same or different and are each a methyl, acetyl or hydrogen radical.

9. A composition comprising a culture medium of an isolated endophyte, wherein the endophyte is a strain of Priestia aryabhattai with Accession Number DSM 34352.

10. A method of producing a composition for bioprotection and/or biostimulant, the method comprising combining:
an isolated endophyte, wherein the endophyte is a strain of Priestia aryabhattai, and
an adjuvant, wherein the strain of Priestia aryabhattai has Accession Number DSM 34352.

11. The method according to claim 10, wherein the adjuvant is selected from the group consisting of (A), (B) or (C):
(A) a polyglycerol ester with a general formula (I) of, 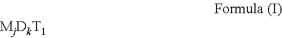
wherein, M is [C₃H₅(OR″)₂O_{1/2}],
D is [C₃H₅(OR″)₁O_{2/2}],
T is [C₃H₅O_{3/2}],
j=1 to 10, preferably 2 to 3, more preferably 2;
k=0 to 10, preferably greater than 0 to 5, more preferably 1 to 3;
l=0 to 3, preferably 0 to 1, more preferably 0;
wherein, the sum total of j+k+l is 1 to 20, preferably 2 to 4, more preferably 3
wherein the radicals R″ are each independently selected from the group consisting of acyl radicals R′—C(═O)— and H, with the proviso that at least one radical R″ is not equal to H;
wherein the radicals R′ are each independently selected from the group consisting of monovalent aliphatic, saturated or unsaturated hydrocarbon radicals with 3 to 39, preferably 7 to 21 more preferably with 9 to 17 carbon atoms;
(B) polyether-modified siloxanes of formula (II) 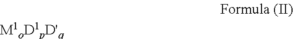
wherein, M¹ is P¹₃SiO_{1/2},
D¹ is P¹₂SiO_{2/2}, D′ is P¹P²SiO_{2/2},
o is 2, p is between 0 and 0.1, q is between 1.0 and 1.15,
P¹ are independently hydrocarbyl having 1 to 8 carbon atoms,
P² are independently a polyether radical of the formula (III) 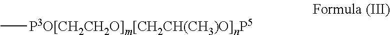
where
m is from 3.4 to 11.0, n is from 2.5 to 8.0,
and with the provisos that
m/n is from 1.9 to 2.8,
P³ are independently divalent hydrocarbyl radicals having 2 to 8 carbon atoms,
P⁵ is hydrogen; and/or
(C) organomodified polysiloxane of formula (IV) 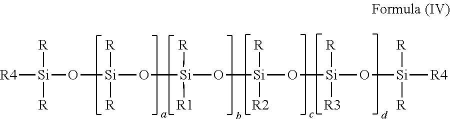
where a+b+c+d+2=20 to 210,
a is 15 to 205, b is 1 to 12, c is 1 to 12, dis 1 to 12,
the R radicals are each independently identical or different, aliphatic or aromatic hydrocarbyl radicals having 1 to 10 carbon atoms,
the R4 radicals are each independently identical or different R, R1, R2 or R3 radicals,
the R1, R2 and R3 radicals are each independently different polyether radicals of general formula (V) 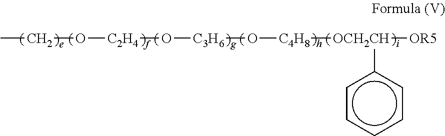
where
e is 3 to 11, f is 6 to 30, g is 0 to 15, h is 0 to 5, i is 0 to 5, and
R5 are independently the same or different and are each a methyl, acetyl or hydrogen radical.

12. A method of providing bioprotection to a plant or part thereof, the method comprising contacting the composition according to claim 1 to the plant or part thereof.

13. Use of the composition according to claim 1 to provide bioprotection to a plant or part thereof.
